(19) United States
(12) Patent Application Publication
(10) Pub. No.: US 2010/0267795 A1
(43) Pub. Date: Oct. 21, 2010

(54) NITROSUBSTITUTED ARYLOXYALKYLIMIDAZOLINES FOR USE AS PESTICIDES

(75) Inventors: Christoph Luethy, Munchenstein (CH); Anthony Cornelius O'Sullivan, Stein (CH); Thomas Pitterna, Stein (CH); Jurgen Harry Schaetzer, Stein (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/741,741

(22) PCT Filed: Oct. 24, 2008
(86) PCT No.: PCT/GB2008/003603

§ 371 Date: May 6, 2010

(30) Foreign Application Priority Data
Nov. 6, 2007 (GB) 0721772.2
Mar. 14, 2008 (GB) 0804782.1
Jun. 26, 2008 (GB) 0811747.5

(51) Int. Cl. A01N 43/50; C07D 233/22; C07C 255/16; A01P 7/02; A01P 7/04

(52) U.S. Cl. 514/401; 548/353.1; 558/389

(57) ABSTRACT

The present invention relates to imidazoline derivatives and their use as insecticidal and acaricidal agents. The invention also extends to insecticidal and acaricidal compositions comprising such imidazoline derivatives, and to methods of using such derivatives and/or compositions to combat and control insect and acarine pests. In particular the present invention relates to phenoxy-imidazoline derivatives wherein the phenoxy moiety is substituted with a nitro group. A method of combating and/or controlling an insect or acarid pest, which comprises applying to said pest, or to the locus of said pest, or to a plant susceptible to attack by said pest, a compound of formula (I): or salt or N-oxide thereof.

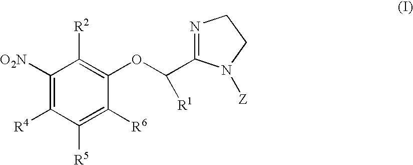

The present invention relates to imidazoline derivatives and their use as insecticidal and acaricidal agents. The invention also extends to insecticidal and acaricidal compositions comprising such imidazoline derivatives, and to methods of using such derivatives and/or compositions to combat and control insect and acarine pests. In particular the present invention relates to phenoxy-imidazoline derivatives wherein the phenoxy moiety is substituted with a nitro group.

A number of imidazoline derivatives are known; for example, U.S. Pat. No. 4,523,020 describes aryloxyarylimidazolines as potential antidiarrheals and analgesics and discloses several nitro-substituted-aryloxy-alkylimidazoline derivatives as synthetic intermediates in their production. DE3404401 describes aryloxy and quinolinyloxy derivatives, several of which also comprise imidazoline moieties, as herbicidal safeners.

Further substituted phenoxy-imidazoline derivatives have been described as being useful in combating ectoparasites and/or acarids: see for example U.S. Pat. No. 4,226,876, U.S. Pat. No. 4,414,223, DE 2818367, EP 0011596, U.S. Pat. No. 4,276,302, U.S. Pat. No. 4,232,011, U.S. Pat. No. 4,241,075, U.S. Pat. No. 4,233,306. However, none of the disclosed compounds bear a nitro-substituted phenoxy moiety.

Whilst Japanese Patent Application No. JP 51106739 describes the compound 2-(4-nitrophenoxymethyl)-2-imidazoline hydrochloride and tests its efficacy at (i) inhibiting oviposition in Boophilus microplus and (ii) killing mosquito larvae, the compound 2-(3-nitro-2-methylphenoxy-methyl)-2-imidazoline hydrochloride is mentioned in U.S. Pat. No. 5,128,361 as a potential animal-ectoparasiticide.

We have now found that further nitrophenoxy-imidazoline derivatives, in particular those where the phenoxy-moiety is substituted at the 3-position with a nitro group, have surprisingly good insecticidal and/or acaricidal activity. Thus according to a first aspect of the invention there is provided a method of combating and controlling insect or acarid pest, which comprises applying to said pest, to a locus of said pest, or to a plant susceptible to attack by said, pest a compound of formula (I):

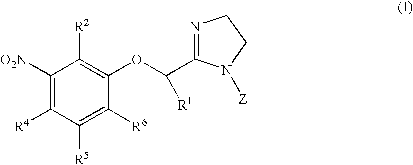

or salt or N-oxide thereof, wherein

R¹ is (i) an optionally substituted C₁₋₆ alkyl, (ii) an optionally substituted C₂₋₆ alkenyl, (iii) an optionally substituted C₃₋₆ cycloalkyl, (iv) an optionally substituted C₃₋₆ cycloalkenyl, or (v) an optionally substituted C₂₋₆ alkynyl;

R² is C₁₋₅ alkyl, C₁₋₅ haloalkyl, C₂₋₆ alkenyl, C₂₋₆ haloalkenyl, alkynyl, C₃₋₆ cycloalkyl, C₁₋₅ alkoxy, C₁₋₅ haloalkoxy, C₁₋₃ alkoxy(C₁₋₃)alkyl, C₁₋₆ alkylthio, C₁₋₅ haloalkylthio, C₁₋₆ alkylsulfonyl, C₁₋₆ alkylsulfinyl, C₁₋₆ haloalkylsulfonyl, C₁₋₅ haloalkylsulfinyl, cyano, nitro, halogen, or formyl;

R⁴ is hydrogen, methyl or halogen;

R⁵ is hydrogen, methyl or halogen;

R⁶ is hydrogen, methyl, or halogen;

Z is hydrogen, hydroxy, nitro, cyano, rhodano, formyl, G-, G-S—, G-S—S—, G-A-, G-O—, G-A-O—, G-X-A-O—, R⁷R⁸N—, R⁷R⁸N-A-, G-O-A-, G-S-A-, (R¹⁰O)(R¹¹O)P(X)—, (R¹⁰O)(R¹¹S)P(X)—, (R¹⁰S)(R¹¹S)P(X)—, (R¹⁰O)(R¹⁴R¹⁵N)P(X)—, (R¹¹)(R¹⁴R¹⁵N)P(X)—, (R¹⁴R¹⁵N)(R¹⁶R¹⁷N)P(X)—, G-N═CH—, G-O—N═CH—, N—N≡CH—, or Z is group of formula (II)

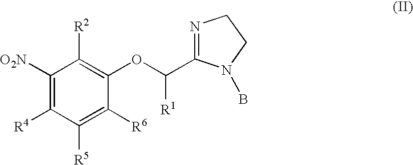

wherein B is S—, S—S—, S(O)—, C(O)—, or (CH₂)ₙ—, n is an integer from 1 to 6; R¹, R², R⁴, R⁵ and R⁶ are as defined above, and

G is optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted C₃₋₇ cycloalkenyl, optionally substituted aryl, optionally substituted heteroaryl or optionally substituted heterocyclyl;

A is S(O), SO₂, C(O) or C(S);

R⁷ and R⁸ are each independently hydrogen or G; or R⁷ and R⁸ together with the N atom to which they are attached form a group N═CR¹²R¹³; or or R⁷ and R⁸ together with the N atom to which they are attached form a five, six or seven-membered heterocyclic ring, which heterocyclic ring optionally contains one or two further heteroatoms selected from O, N or S, and is optionally substituted by one or two C₁₋₆ alkyl groups;

R¹⁰ and R¹¹ are each independently C₁₋₆ alkyl, benzyl or phenyl where the phenyl group is optionally substituted with halogen, nitro, cyano, C₁₋₃ alkyl, C₁₋₃ haloalkyl, C₁₋₃ alkoxy, C₁₋₃ haloalkoxy;

R¹², R¹³, R¹⁴, R¹⁵, R¹⁶ and R¹⁷ are each independently hydrogen or C₁₋₆ alkyl;

X is O or S.

For the avoidance of doubt, the term “compound” as used herein includes all salts and N-oxides of said compound.

The compounds of formula (I) as described herein may exist in different geometric or optical isomers or different tautomeric forms. One or more centres of chirality may be present, for example on the chiral carbon atom CHR¹ or a chiral carbon unit in the group G, or a chiral —S(O)— unit, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers or mixtures of isomers. Centres of tautomerisation may be present. This invention covers all such isomers and tautomers and mixtures thereof in all proportions as well as isotopic forms such as deuterated compounds.

Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as G, alkoxy, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. The alkyl groups are suitably C₁ to C₁₀ alkyl groups, but are preferably C₁-C₈, even more preferably C₁-C₆ and most preferably C₁-C₄ alkyl groups.

Ring or chain forming alkylene, alkenylene and alkinylene groups can optionally be further substituted by one or more halogen, C₁₋₃ alkyl and/or C₁₋₃ alkoxy group.

When present and unless explicitly stated herein to the contrary, the optional substituents on an alkyl moiety (alone or as part of a larger group such as G, alkoxy, alkoxycarbonyl, alkylcarbonyl, alkylaminocarbonyl, dialkylaminocarbonyl) include one or more of halogen, nitro, cyano, rhodano, isothiocyanato, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkylsilyl(C₁₋₆)alkoxy, C₁₋₆ alkoxycarbonyl(C₁₋₁₀)alkoxy, C₁₋₁₀ haloalkoxy, aryl(C₁₋₄)-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄)alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryldi(C₁₋₄)alkylsilyl, triarylsilyl, aryl(C₁₋₄)alkylthio(C₁₋₄)alkyl, aryloxy(C₁₋₄alkyl, formyl, C₁₋₁₀ alkylcarbonyl, HO₂C, C₁₋₁₀ alkoxycarbonyl, aminocarbonyl, C₁₋₆ alkylaminocarbonyl, di(C₁₋₆alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes and oximethers such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that allenyl and alkinylalkenyl are included in these terms.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl, optionally substituted C₂₋₆ alkynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂C₁, CF₂H, CCl₂H, FCH₂, ClCH₂, BrCH₂, CH₃CHF, (CH₃)₂CF, CF₃CH₂ or CHF₂CH₂.

Haloalkenyl groups are alkenyl groups which are substituted with one or more of the same or different halogen atoms.

In the context of the present specification the terms “aryl”, “aromatic ring” and “aromatic ring system” refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

When present, the optional substituents on aryl or heteroaryl are selected independently, from halogen, nitro, cyano, rhodano, isothiocyanato, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₆₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxy, Carbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄)-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include aryl carbonyl amino (where the aryl group is substituted by C₁₋₆ alkyl or halogen), C₁₋₆alkoxycarbonylamino, C₁₋₆alkoxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroaryl amino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆ alkylaminocarbonyl amino, di(C₁₋₆)alkylaminocarbonyl amino, arylaminocarbonyl amino where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆alkylaminocarbonyl-N—(C₁₋₆)alkyl amino, di(C₁₋₆)alkylaminocarbonyl-N—(C₁₋₆)alkyl amino, arylaminocarbonyl-N—(C₁₋₆)alkyl amino (where the aryl group is substituted by C₁₋₆ alkyl or halogen) and aryl-N—(C₁₋₆)alkylaminocarbonyl-N—(C₁₋₆)alkyl amino (where the aryl group is substituted by C₁₋₆ alkyl or halogen).

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholin and piperazine.

When present, the optional substituents on heterocyclyl include C₁₋₆ alkyl and C₁₋₆ haloalkyl, an oxo-group (wherein one of the carbon atoms in the ring may be in the form of a keto group), as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, amino, C₁₋₆ alkylamino, di(C₁₋₆ alkyl)amino, aminocarbonyl, C₁₋₆ alkylaminocarbonyl, or di(C₁₋₆ alkyl)aminocarbonyl.

Preferably the optional substituents on an alkyl moiety include one or more of halogen, nitro, cyano, HO₂C, C₁₋₁₀ alkoxy (itself optionally substituted by C₁₋₁₀ alkoxy), aryl(C₁₋₄)alkoxy, C₁₋₁₀ alkylthio, C₁₋₁₀ alkylcarbonyl, cycloalkylcarbonyl, C₁₋₁₀ alkoxycarbonyl, C₁₋₆ alkylaminocarbonyl, di(C₁₋₆ alkyl)aminocarbonyl, (C₁₋₆)alkylcarbonyloxy, optionally substituted phenyl, heteroaryl, aryloxy, arylcarbonyloxy, heteroaryloxy, heterocyclyl, heterocyclyloxy, C₃₋₇ cycloalkyl (itself optionally substituted with (C₁₋₆)alkyl or halogen), C₃₋₇ cycloalkyloxy, C₅₋₇ cycloalkenyl, C₁₋₆ alkylsulfonyl, C₁₋₆ alkylsulfinyl, tri(C₁₋₄alkylsilyl(C₁₋₆)alkoxy, aryldi(C₁₋₄alkylsilyl, (C₁₋₄)alkyldiarylsilyl and triarylsilyl.

Preferably the optional substituents on alkenyl or alkynyl include one or more of halogen, aryl and C₃₋₇ cycloalkyl.

A particularly preferred optional substituent for heterocyclyl is C₁₋₃ alkyl.

Preferably the optional substituents for cycloalkyl include halogen, cyano and C₁₋₆ alkyl.

The optional substituents for cycloalkenyl preferably include C₁₋₃ alkyl, halogen and cyano.

In particularly preferred embodiments of the invention, the preferred groups for R¹, R², R⁴, R⁵, R⁶, and Z, in any combination thereof, are as set out below.

In certain embodiments, where R¹ is substituted, said substitution is preferably selected from one or more of: halogen; nitro; cyano; rhodano; carboxy; formyl; formyloxy; formylamino; optionally substituted C₃₋₇ cycloalkyl, said substitution being selected from one or more of halogen, hydroxy, nitro, cyano, rhodano, C₁₋₃ alkyl, C₁₋₃ haloalkyl and C₁₋₃ alkoxy; optionally substituted C₃₋₇ cycloalkenyl, said substitution being selected from one or more of halogen, hydroxy, nitro, cyano, rhodano, C₁₋₃ alkyl, C₁₋₃ haloalkyl, and C₁₋₃ alkoxy; optionally substituted aryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, and C₁₋₄ alkoxycarbonyl; optionally substituted heteroaryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, C₁₋₃ alkoxycarbonyl, carbamoyl, C₁₋₄ alkylaminocarbonyl and di-C₁₋₄ alkylaminocarbonyl; optionally substituted heterocyclyl, said substitution being selected from one or more of halogen, hydroxy, nitro, cyano, rhodano, C₁₋₃ alkyl, C₁₋₃ haloalkyl, and C₁₋₃ alkoxy; G-O—; G-S—; G-A-; G-A-O—; G-A-S—; R⁷R⁸N—; R⁷R⁸N-A-; G-O-A-; G-S-A-; G-A-NR⁹—; R⁷R⁸N-A-NR⁹—; and G-O-A-NR⁹—, wherein G, R⁷, R⁸, A and R⁹ are as defined hereinbefore.

In preferred embodiments when R¹ is a C₁₋₆ alkyl moiety, it is optionally substituted with one or more of: halogen, cyano, optionally substituted C₃₋₆ cycloalkyl (said substitution being selected from halogen, cyano, C₁₋₃ haloalkyl and C₁₋₃ alkoxy), optionally substituted pyridyl, pyrimidinyl, furyl or thienyl (said substitution being selected from halogen, cyano, C₁₋₃ alkyl, C₁₋₃ haloalkyl and C₁₋₃ alkoxy), optionally substituted tetrahydrofuryl, tetrahydropyranyl or 1,3-dioxolanyl (said substitution being selected from C₁₋₃ alkyl and C₁₋₃ alkoxy), C₁₋₃ alkoxy, C₁₋₃ haloalkoxy, allyloxy, propargyloxy, C₁₋₃ alkylthio, C₁₋₃ alkylsulfinyl, C₁₋₃ alkylsulfonyl, formyl, C₁₋₃ alkylcarbonyl, cyclopropylcarbonyl, C₁₋₃ haloalkylcarbonyl, C₁₋₃ alkoxycarbonyl, C₁₋₃ alkylthiocarbonyl, aminocarbonyl, C₁₋₃ alkylaminocarbonyl, N,N-di-(C₁₋₃alkyl)aminocarbonyl, amino, C₁₋₃ alkylamino, di (C₁₋₃alkyl)amino, formylamino, C₁₋₃alkylcarbonylamino, C₁₋₃ haloalkylcarbonylamino, C₁₋₃ alkylsulfonylamino, C₁₋₃ haloalkylsulfonylamino, C₁₋₃ alkylcarbonyloxy, C₁₋₃ haloalkylcarbonyloxy, cyclopropylcarbonyloxy, benzoyloxy, C₁₋₃ alkoxycarbonylamino, C₁₋₃ alkoxycarbonyloxy and C₁₋₃ alkylcarbonylthio.

In other preferred embodiments R¹ is a C₂₋₆ alkenyl moiety, optionally substituted with one or more of: halogen, cyano, optionally substituted C₃₋₆ cycloalkyl, (said substitution being selected from halogen and C₁₋₃ alkoxy), C₁₋₃ alkoxy, C₁₋₃ haloalkoxy, C₁₋₃ alkylthio, C₁₋₃ haloalkylthio, C₁₋₃ alkylsulfinyl, C₁₋₃ alkylsulfonyl, C₁₋₃ alkylcarbonyl, C₁₋₃ alkoxycarbonyl, aminocarbonyl, C₁₋₃ alkylaminocarbonyl, N,N-di-(C₁₋₃ alkyl)aminocarbonyl, amino, C₁₋₃ alkylamino, formylamino, C₁₋₃alkylcarbonylamino, C₁₋₃ haloalkylcarbonylamino, and C₁₋₃ alkylsulfonylamino.

In further preferred embodiments R¹ is a C₃₋₆ cycloalkyl moiety optionally substituted with halogen, C₁₋₃ alkyl, C₁₋₃ haloalkyl, or C₁₋₃ alkoxy; C₃₋₆ cycloalkenyl optionally substituted with halogen, C₁₋₃ alkyl, C₁₋₃ haloalkyl, or C₁₋₃ alkoxy; C₂₋₆ alkynyl optionally substituted with halogen, hydroxyl, cyano, C₁₋₃ alkoxy, C₁₋₃ haloalkoxy, or C₁₋₃ alkylthio. In still other preferred embodiments R¹ is C₃₋₆ cycloalkenyl optionally substituted with one or more of halogen, C₁₋₃ alkyl, C₁₋₃ haloalkyl, or C₁₋₃ alkoxy, or R¹ is C₂₋₆ alkynyl optionally substituted with one or more of halogen, hydroxyl, cyano, C₁₋₃ alkoxy, C₁₋₃ haloalkoxy, or C₁₋₃ alkylthio.

In particularly preferred embodiments R¹ is: ethyl; or propyl (n-propyl or iso-propyl); or butyl; or C₁₋₃ alkyl substituted with halogen, cyano, cyclopropyl, C₁₋₃ alkoxy, allyloxy, propargyloxy, C₁₋₃ alkylthio, C₁₋₃ alkylsulfinyl, C₁₋₃ alkylsulfonyl, formyl, C₁₋₃ alkylcarbonyl, cyclopropylcarbonyl, C₁₋₃ haloalkylcarbonyl, C₁₋₃ alkoxycarbonyl, C₁₋₃ alkylthiocarbonyl, aminocarbonyl, C₁₋₃ alkylaminocarbonyl, N,N-di-(C₁₋₃ alkyl)aminocarbonyl, C₁₋₃ alkylcarbonyloxy, C₁₋₃ haloalkylcarbonyloxy, cyclopropylcarbonyloxy, benzoyloxy, or C₁₋₃ alkoxycarbonyloxy; or C₂₋₄ alkenyl, optionally substituted with halogen, cyano, C₁₋₃ alkoxy, C₁₋₃ haloalkoxy, C₁₋₃ alkylthio, C₁₋₃ haloalkylthio, C₁₋₃ alkylsulfinyl, C₁₋₃ alkylsulfonyl, C₁₋₃ alkylcarbonyl, or C₁₋₃ alkoxycarbonyl; or C₂₋₄ alkynyl optionally substituted with halogen, cyano, or C₁₋₃ alkoxy; or C₃₋₆ cycloalkyl optionally substituted with halogen, or C₁₋₃ alkyl.

In more preferred embodiments R¹ is: ethyl; n-propyl; C₁₋₃ alkyl substituted with methoxy; or allyl, optionally substituted with halo, cyano, or C₁₋₂ alkoxy; or cyclopropyl, optionally substituted with halogen or C₁₋₂ alkyl. In the most preferred embodiments R¹ is ethyl or propyl.

In certain embodiments, including any of those described hereinbefore, Z is hydrogen, cyano, formyl, C₁₋₆ alkyl [optionally substituted by 1-7 fluorine atoms, 1-3 chlorine atoms, 1-3 bromine atoms, a cyano group, 1-2 C₁₋₃ alkoxy groups, a C₁₋₃ haloalkoxy group, a C₁₋₃ alkylthio group, a C₁₋₃ haloalkylthio group, an allyloxy group, a propargyloxy group, a C₃₋₆ cycloalkyl group, phenyl (itself optionally substituted by halogen, nitro, cyano, C₁₋₃ alkyl, C₁₋₃ alkoxy), a C₁₋₃ alkylcarbonyloxy group, a C₁₋₃ alkoxycarbonyl group, a C₁₋₃ alkylcarbonyl group, benzoyl (itself optionally substituted by halogen, nitro, C₁₋₃ alkyl, C₁₋₃ alkoxy, or a cyano group)], C₃₋₆ alkenyl, C₃₋₆ haloalkenyl, C₃₋₆ alkinyl, C₁₋₆ alkylthio, C₁₋₆ haloalkylthio, C₁₋₆ cyanoalkylthio, phenylthio (optionally substituted by halogen, nitro, cyano, C₁₋₃ alkyl, or C₁₋₃ alkoxy), C₁₋₆ alkyldithio, di(C₁₋₄ alkyl)aminothio, C₁₋₆ alkylcarbonyl (optionally substituted by halogen, cyano, or C₁₋₃ alkoxy), C₂₋₆ alkenylcarbonyl, C₃₋₆ cycloalkylcarbonyl, phenylcarbonyl (optionally substituted by halogen, nitro, cyano, C₁₋₃ alkyl, or C₁₋₃ alkoxy), heteroarylcarbonyl (optionally substituted by halogen, nitro, cyano, C₁₋₃ alkyl, or C₁₋₃ alkoxy), C₁₋₆ alkoxycarbonyl, C₁₋₆ alkylthio-carbonyl, phenylthio-carbonyl (optionally substituted by halogen, nitro, cyano, C₁₋₃ alkyl, or C₁₋₃ alkoxy), N,N-di C₁₋₃ alkylaminocarbonyl, C₁₋₃ alkylaminocarbonyl, C₃₋₅ alkenylaminocarbonyl, C₃₋₅ alkynylaminocarbonyl, phenylaminocarbonyl (in which the phenyl group can be optionally substituted by halogen, nitro, cyano, C₁₋₃ alkyl, or C₁₋₃ alkoxy), N-phenyl-N-methyl aminocarbonyl (in which the phenyl group can be optionally substituted by halogen, nitro, cyano, C₁₋₃ alkyl, or C₁₋₃ alkoxy), C₁₋₆ alkoxythionocarbonyl, C₁₋₆ alkylthiothionocarbonyl, phenylthiothionocarbonyl (optionally substituted by halogen, nitro, cyano, C₁₋₃ alkyl, or C₁₋₃ alkoxy), N,N-di C₁₋₃ alkylaminothionocarbonyl, C₁₋₃ alkylaminothionocarbonyl, phenylaminothionocarbonyl (in which the phenyl group can be optionally substituted by halogen, nitro, cyano, C₁₋₃ alkyl, or C₁₋₃ alkoxy), N-phenyl-N-methyl aminothionocarbonyl (in which the phenyl group can be optionally substituted by halogen, nitro, cyano, C₁₋₃ alkyl, or C₁₋₃ alkoxy), C₁₋₃ alkylsulfonyl, C₁₋₃ haloalkylsulfonyl, C₁₋₃ alkenylsulfonyl, phenylsulfonyl (optionally substituted by halogen, nitro, cyano, C₁₋₃ alkyl, or C₁₋₃ alkoxy), N,N-di C₁₋₃ alkylaminosulfonyl, di C₁₋₃ alkoxy-P(═O)—, di C₁₋₃ alkylthio-P(═O)—, di C₁₋₃ alkoxy-P(═S)—, di C₁₋₃ alkylthio-P(═S)—, (C₁₋₃ alkoxy)(phenyl)(P═O)—, (C₁₋₃ alkoxy)(phenyl)(P═S)—, C₁₋₃ alkyl-N═CH—, C₁₋₃ alkoxy-N═CH—, cyano-N═CH—, phenyl-N═CH— (in which the phenyl is optionally substituted by halogen, nitro, cyano, C₁₋₃ alkyl, or C₁₋₃ alkoxy), 2-pyridyl-N═CH—, 3-pyridyl-N═CH—, 2-thiazolyl-N═CH—, or a compound of formula (II) as described hereinbefore wherein B is S—, or CH₂—.

More preferably Z is hydrogen, cyano, formyl, C₁₋₃ alkyl, C₁₋₃ haloalkyl, C₁₋₃ cyanoalkyl, C₁₋₃ alkoxy-C₁₋₃ alkyl, C₁₋₃ benzoyloxy-C₁₋₃ alkyl, allyl, propargyl, C₁₋₆ alkylthio, C₁₋₆ haloalkylthio, phenylthio (optionally substituted by halogen, C₁₋₃ alkyl or C₁₋₃ alkoxy), C₁₋₆ alkylcarbonyl, phenylcarbonyl (optionally substituted by halogen, C₁₋₃ alkyl or C₁₋₃ alkoxy), C₁₋₆ alkoxycarbonyl, C₁₋₃ alkylaminocarbonyl, phenylaminocarbonyl (in which the phenyl group can be optionally substituted by halogen, C₁₋₃ alkyl or C₁₋₃ alkoxy), C₁₋₃ alkylaminothionocarbonyl, phenylaminothionocarbonyl (in which the phenyl group can be optionally substituted by halogen, C₁₋₃ alkyl or C₁₋₃ alkoxy), C₁₋₃ alkylsulfonyl, C₁₋₃ haloalkylsulfonyl, di(C₁₋₃ alkoxy)-P(═O)—, C₁₋₃ alkoxy-N═CH—, cyano-N═CH—, 2-pyridyl-N═CH—. Even more preferably Z is hydrogen or C(O)O-t-C₄H₉. Most preferably Z is hydrogen.

In preferred embodiments, R² is fluoro, chloro, bromo, C₁₋₂ alkyl or C₁₋₂ haloalkyl. More preferably R² is methyl, fluoromethyl, difluoromethyl, trifluoromethyl, fluoro, chloro, or bromo. Most preferably R² is methyl, fluoro, chloro or bromo.

In preferred embodiments R⁴ is hydrogen or fluoro, more preferably hydrogen.

In preferred embodiments R⁵ is hydrogen, methyl, chloro or fluoro, more preferably hydrogen or fluoro. Most preferably R⁵ is hydrogen.

In preferred embodiments R⁶ is hydrogen, methyl, chloro or fluoro, more preferably hydrogen or fluoro. Most preferably R⁶ is hydrogen.

In particularly preferred embodiments, each of R⁴, R⁵ and R⁶ is hydrogen.

Certain compounds of formula (I) are novel and as such they form a further aspect of the invention. These novel compounds are referred to herein as compounds of formula (IA). Thus the invention also provides a compound of formula (IA)

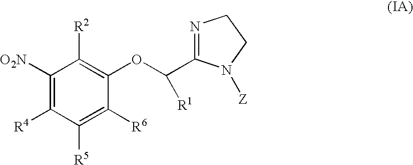

or salt or N-oxide thereof, wherein:

R¹ is (i) substituted C₁₋₂ alkyl provided said substitution is not a hydroxyl group (ii) optionally substituted C₃₋₆ alkyl, (iii) optionally substituted C₂₋₆ alkenyl, (iv) optionally substituted C₃₋₆ cycloalkyl, (v) optionally substituted C₃₋₆ cycloalkenyl, or (vi) optionally substituted C₂₋₆ alkynyl; and R², R⁴, R⁵, R⁶ and Z are as defined hereinbefore.

In this aspect of the invention the preferred groups for R², R⁴, R⁵, R⁶ and Z are as set out above in respect of compounds of formula (I) and in the illustrative compounds described below, whilst the preferred groups for R¹ are as set out below. The skilled man will appreciate that the invention encompasses any combination of these preferred groups.

In certain embodiments of compounds of formula (IA), where R¹ is substituted, said substitution is preferably selected from one or more of: halogen; nitro; cyano; rhodano; carboxy; formyl; formyloxy; formylamino; optionally substituted C₃₋₇ cycloalkyl, said substitution being selected from one or more of halogen, hydroxy, nitro, cyano, rhodano, C₁₋₃ alkyl, C₁₋₃ haloalkyl and C₁₋₃ alkoxy; optionally substituted C₃₋₇ cycloalkenyl, said substitution being selected from one or more of halogen, hydroxy, nitro, cyano, rhodano, C₁₋₃ alkyl, C₁₋₃ haloalkyl, and C₁₋₃ alkoxy; optionally substituted aryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, and C₁₋₄ alkoxycarbonyl; optionally substituted heteroaryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, C₁₋₃ alkoxycarbonyl, carbamoyl, C₁₋₄ alkylaminocarbonyl and di-C₁₋₄ alkylaminocarbonyl; optionally substituted heterocyclyl, said substitution being selected from one or more of halogen, hydroxy, nitro, cyano, rhodano, C₁₋₃ alkyl, C₁₋₃ haloalkyl, and C₁₋₃ alkoxy; G-O—; G-S—; G-A-; G-A-O—; G-A-S—; R⁷R⁸N—; R⁷R⁸N-A-; G-O-A-; G-S-A-; G-A-NR⁹—; R⁷R⁸N-A-NR⁹—; and G-O-A-NR⁹—, wherein G, R⁷, R⁸, A and R⁹ are as defined hereinbefore.

In preferred embodiments of compounds of formula (IA) R¹ is an unsubstituted C₃₋₆ alkyl moiety or a substituted C₁₋₆alkyl moiety wherein said substitution is selected from: halogen, cyano, optionally substituted C₃₋₆ cycloalkyl (said substitution being selected from halogen, cyano, C₁₋₃ haloalkyl and C₁₋₃ alkoxy), optionally substituted pyridyl, pyrimidinyl, furyl or thienyl (said substitution being selected from halogen, cyano, C₁₋₃ alkyl, C₁₋₃ haloalkyl and C₁₋₃ alkoxy), optionally substituted tetrahydrofuryl, tetrahydropyranyl or 1,3-dioxolanyl (said substitution being selected from C₁₋₃ alkyl and C₁₋₃ alkoxy), C₁₋₃ alkoxy, C₁₋₃ haloalkoxy, allyloxy, propargyloxy, C₁₋₃ alkylthio, C₁₋₃ alkylsulfinyl, C₁₋₃ alkylsulfonyl, formyl, C₁₋₃ alkylcarbonyl, cyclopropylcarbonyl, C₁₋₃ haloalkylcarbonyl, C₁₋₃ alkoxycarbonyl, C₁₋₃ alkylthiocarbonyl, aminocarbonyl, C₁₋₃ alkylaminocarbonyl, N,N-di-(C₁₋₃alkyl)aminocarbonyl, amino, C₁₋₃ alkylamino, di (C₁₋₃ alkyl)amino, formylamino, C₁₋₃alkylcarbonylamino, C₁₋₃ haloalkylcarbonylamino, C₁₋₃ alkylsulfonylamino, C₁₋₃ haloalkylsulfonylamino, C₁₋₃ alkylcarbonyloxy, C₁₋₃ haloalkylcarbonyloxy, cyclopropylcarbonyloxy, benzoyloxy, C₁₋₃ alkoxycarbonylamino, C₁₋₃ alkoxycarbonyloxy and C₁₋₃ alkylcarbonylthio.

In other preferred embodiments of compounds of formula (IA) R¹ is a C₂₋₆ alkenyl moiety, optionally substitiited with one or more of: halogen, cyano, optionally substituted C₃₋₆ cycloalkyl, (said substitution being selected from halogen and C₁₋₃ alkoxy), C₁₋₃ alkoxy, C₁₋₃ haloalkoxy, C₁₋₃ alkylthio, C₁₋₃ haloalkylthio, C₁₋₃ alkylsulfinyl, C₁₋₃ alkylsulfonyl, C₁₋₃ alkylcarbonyl, C₁₋₃ alkoxycarbonyl, aminocarbonyl, C₁₋₃ alkylaminocarbonyl, N,N-di-(C₁₋₃alkypaminocarbonyl, amino, C₁₋₃ alkylamino, formylamino, C₁₋₃alkylcarbonylamino, C₁₋₃ haloalkylcarbonylamino, and C₁₋₃ alkylsulfonylamino.

In further preferred embodiments of compounds of formula (IA) R¹ is a C₃₋₆ cycloalkyl moiety optionally substituted with halogen, C₁₋₃ alkyl, C₁₋₃ haloalkyl, or C₁₋₃ alkoxy; C₃₋₆ cycloalkenyl optionally substituted with halogen, C₁₋₃ alkyl, C₁₋₃ haloalkyl, or C₁₋₃ alkoxy; C₂₋₆ alkynyl optionally substituted with halogen, hydroxyl, cyano, C₁₋₃ alkoxy, C₁₋₃ haloalkoxy, or C₁₋₃ alkylthio.

In particularly preferred embodiments of compounds of formula (IA) R¹ is: propyl (n-propyl or iso-propyl); butyl; C₁₋₃ alkyl substituted with one or more of halogen, cyano, cyclopropyl, C₁₋₃ alkoxy, allyloxy, propargyloxy, C₁₋₃ alkylthio, C₁₋₃ alkylsulfinyl, C₁₋₃ alkylsulfonyl, formyl, C₁₋₃ alkylcarbonyl, cyclopropylcarbonyl, C₁₋₃ haloalkylcarbonyl, C₁₋₃ alkoxycarbonyl, C₁₋₃ alkylthiocarbonyl, aminocarbonyl, C₁₋₃alkylaminocarbonyl, N,N-di-(C₁₋₃alkyl)aminocarbonyl, C₁₋₃ alkylcarbonyloxy, C₁₋₃ haloalkylcarbonyloxy, cyclopropylcarbonyloxy, benzoyloxy, or C₁₋₃ alkoxycarbonyloxy; C₂₋₄ alkenyl optionally substituted with one or more of halogen, cyano, C₁₋₃ alkoxy, C₁₋₃ haloalkoxy, C₁₋₃ alkylthio, C₁₋₃ haloalkylthio, C₁₋₃ alkylsulfinyl, C₁₋₃ alkylsulfonyl, C₁₋₃ alkylcarbonyl, or C₁₋₃ alkoxycarbonyl; C₂₋₄ alkynyl optionally substituted with one or more of halogen, cyano, or C₁₋₃ alkoxy; or cyclopropyl optionally substituted with one or more of halogen or C₁₋₂ alkyl.

In more preferred embodiments of compounds of formula (IA) R¹ is: propyl; C₁₋₃ alkyl substituted with methoxy; allyl optionally substituted with one or more of halogen, cyano, or C₁₋₂ alkoxy; or cyclopropyl optionally substituted with one or more of halogen or C₁₋₂ alkyl. In the most preferred embodiments of compounds of formula (IA) R¹ is n-propyl.

Illustrative examples of compounds of formulae I and IA are described below. Table 1 provides 32 compounds of formula Ia

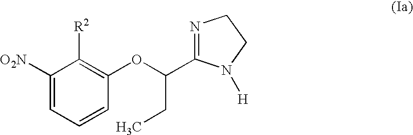

wherein the values of R² are given in the table.

TABLE 1

| Compound No | R² |
|---|---|
| I-1 | —CN |
| I-2 | —NO₂ |
| I-3 | Me |
| I-4 | Et |
| I-5 | Pr |
| I-6 | iPr |
| I-7 | Cl₂C═CH— |
| I-8 | Vinyl |
| I-9 | Allyl |
| I-10 | Propargyl |
| I-11 | CF₃ |
| I-12 | CHF₂ |
| I-13 | CH₂F |
| I-14 | CF₂Cl |
| I-15 | CCl₃ |
| I-16 | Cyclopropyl |
| I-17 | MeO |
| I-18 | F₃CO |
| I-19 | F₂CHO |
| I-20 | MeOCH₂ |
| I-21 | MeS |
| I-22 | F₃CS |
| I-23 | F₂HCS |
| I-24 | F |
| I-25 | Cl |
| I-26 | Br |
| I-27 | I |
| I-28 | F₂ClCO |
| I-29 | MeS(O)— |
| I-30 | MeS(O)₂— |
| I-31 | F₃CS(O)— |
| I-32 | F₃CS(O)₂ |

32 Compounds of formula (Ib),

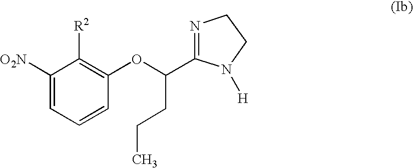

wherein the values of R² are as given in Table 1 for compounds 1-1 to 1-32, are designated as compound Nos. II-1 to II-32, respectively.

32 Compounds of formula (Ic),

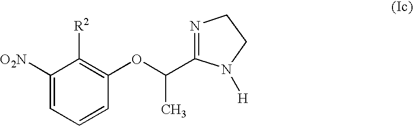

wherein the values of R² are as given in Table 1 for compounds I-1 to I-32, are designated as compound Nos. III-1 to III-32, respectively.

32 Compounds of formula (Id),

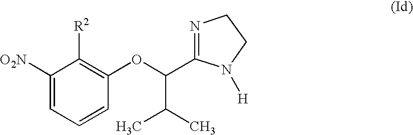

wherein the values of R² are as given in Table 1 for compounds I-1 to I-32, are designated as compound Nos. IV-1 to IV-32, respectively.

32 Compounds of formula (Ie),

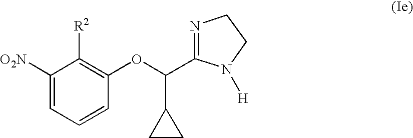

wherein the values of R² are as given in Table 1 for compounds I-1 to I-32, are designated as compound Nos. V-1 to V-32, respectively.

32 Compounds of formula (If),

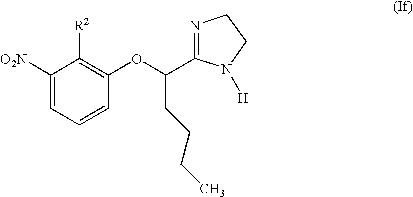

wherein the values of R² are as given in Table 1 for compounds I-1 to I-32, are designated as compound Nos. VI-1 to VI-32, respectively.

32 Compounds of formula (Ig),

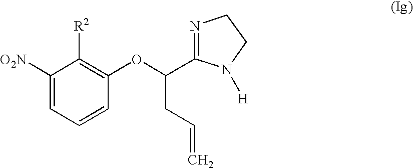

wherein the values of R² are as given in Table 1 for compounds I-1 to I-32, are designated as compound Nos. VII-1 to VII-32, respectively.

32 Compounds of formula (Ih),

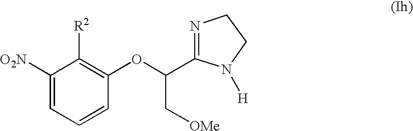

wherein the values of R² are as given in Table 1 for compounds I-1 to I-32, are designated as compound Nos. VIII-1 to VIII-32, respectively.

Table 2 below provides characterising data for compounds of the invention. The skilled man will appreciate that compound numbers 1.001, 1.002, 1,003, 1.004, 1.005, 1.008, 1.009, 1.012, 1.013, 1.014, 1.015, 1.016, 1.017, and 1.018 in Table 2 correspond to compound numbers I-3, IV-3, V-3, II-3, VI-3, I-25, II-25, VIII-3, VII-3, IV-25, V-25, VII-25, VIII-25 and II26 (respectively) as described above.

TABLE 2
Characterising data for compounds of the invention.

| compound | | | | | | | m.p. |
|---|---|---|---|---|---|---|---|
| number | R¹ | R² | R⁴ | R⁵ | R⁶ | Z | [° C.] |
| 1.001 | C₂H₅ | CH₃ | H | H | H | H | 133-135 |
| 1.002 | i-C₃H₇ | CH₃ | H | H | H | H | 97-101 |
| 1.003 | c-C₃H₇ | CH₃ | H | H | H | H | 107-109 |
| 1.004 | n-C₃H₇ | CH₃ | H | H | H | H | 163-165 |
| 1.005 | n-C₄H₉ | CH₃ | H | H | H | H | 109-112 |
| 1.006 | n-C₃H₇ | CH₃ | H | H | H | C(O)O-t-C₄H₉ | 85-87 |
| 1.007 | C₂H₅ | CH₃ | H | H | H | C(O)O-t-C₄H₉ | 97-100 |
| 1.008 | C₂H₅ | Cl | H | H | H | H | 100-103 |
| 1.009 | n-C₃H₇ | Cl | H | H | H | H | 159-160 |
| 1.010 | n-C₃H₇ | Cl | H | H | H | C(O)O-t-C₄H₉ | 125-127 |
| 1.011 | C₂H₅ | Cl | H | H | H | C(O)O-t-C₄H₉ | 114-116 |
| 1.012 | CH₂OMe | CH₃ | H | H | H | H | 65-72 |
| 1.013 | allyl | CH₃ | H | H | H | H | 145-147 |
| 1.014 | i-C₃H₇ | Cl | H | H | H | H | 102-107 |
| 1.015 | c-C₃H₇ | Cl | H | H | H | H | 131-132 |
| 1.016 | allyl | Cl | H | H | H | H | 137-142 |
| 1.017 | CH₂OMe | Cl | H | H | H | H | 106-108 |
| 1.018 | n-C₃H₇ | Br | H | H | H | H | 148-151 |

Compounds of the invention can be prepared by a variety of methods, for example those described below.

Compounds of formula (I) wherein Z is not H can be prepared from compounds of formula (I) in which Z is H, by treatment with the appropriate reagent. Depending on the nature of Z this can be for example an alkylating agent, an acylating agent, a phosphorylating agent, a carbamoylating agent, a sulfenylating agent or an oxidising agent. These derivatisating agents are generally electrophiles. Methods for the conversion of NH groups into NZ groups can be found for example in T. W. Greene and P. G. M. Wuts “Protecting Groups in Organic Synthesis” 3^{rd} Edition, Wiley, NY 1999.

Similarly if the group R¹ contains a reactive moiety, compounds of the formula (I) bearing such an R¹ group can be converted into other compounds of the formula (I), by means of chemical transformation of the reactive moiety in group R¹.

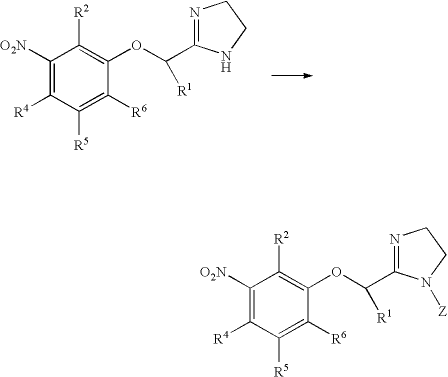

Compounds of formula (I) can be prepared by alkylation of a phenol of the formula 2, with a 2-haloalkylimidazoline of the formula 3 (J. Am. Chem. Soc. 1947, 69, 1688).

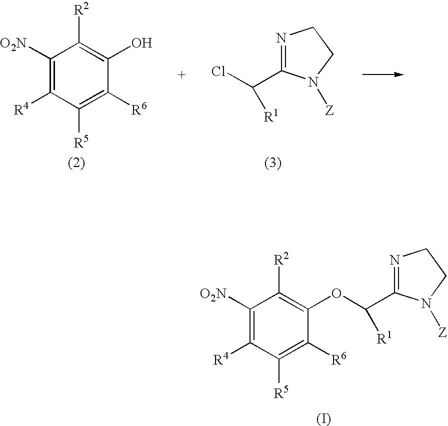

Compounds of the formula (I) can be prepared from nitriles of the formula (4), by treatment with a diamine of the formula (5), wherein Z has the meanings assigned to it above. This is advantageously performed in the presence of a catalyst such as CS₂, P₂S₅ (J. Med. Chem., 2003 46, 1962) or Na₂S₄ (DE 2512513). The nitrile (4) can be converted to imidates of formula (6) using an alcohol such as methanol and a catalytic amount of base such as NaOMe, or to imidate salts of formula (6a) using an alcohol such as methanol or ethanol and an acid such as HCl. Imidates of the formula (6)/(6a) can be converted to compounds of formula (I) on treatment with diamines of formula (5) (J. Med. Chem., 2004, 47, 6160; J: Am. Chem. Soc. 1947, 69, 1688). Nitriles of formula (4) can be prepared by alkylating phenols of formula (2) with a nitrile of formula (9), bearing a leaving group L₁ (J. Am. Chem. Soc. 1947, 69, 1688).

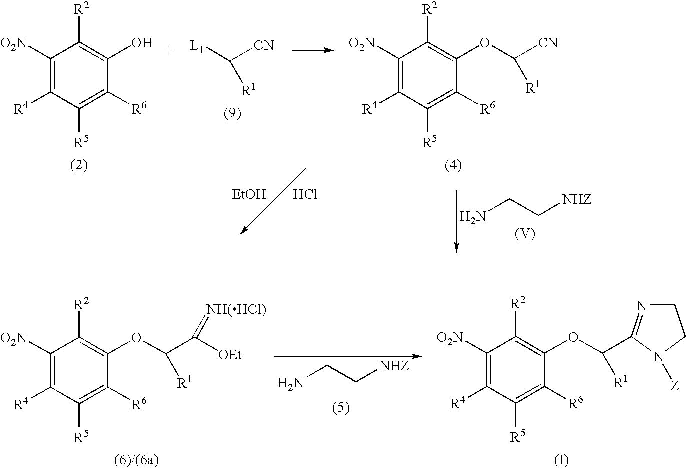

Esters of formula (7) can be converted to imidazolines of formula (I) by treatment with diamines of formula (5) (J. Am. Chem. Soc. 1950, 72, 4443-5). Alkylaluminium reagents can be used with advantage to facilitate this reaction. This conversion occurs in two steps by forming first the monoamide (11), which can serve as a precursor to imidazolines of the formula (I). Esters of formula (7) can be prepared by alkylation of phenols of formula (2) with esters of formula (10), wherein L₂ is a leaving group, and R²¹ is an optionally substituted alkyl or aryl group (typically C₁-C₆ alkyl, phenyl or benzyl). The leaving groups L₁ and L₂ are typically those used for S_{N}2 reactions. L₁ and L₂ become anions of organic or inorganic acids on leaving their substrates (9) and (10). Typical leaving groups include for example, halides such as chloride or bromide, alkylsulfonates such as mesylate, and arylsulfonates such as tosylate.

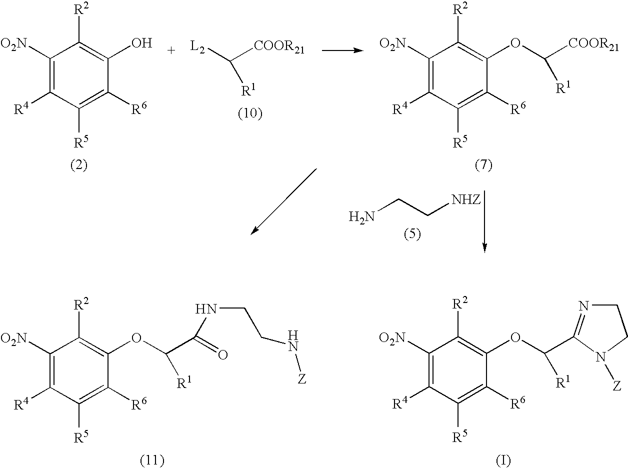

Compounds of formula (I) can be prepared from imidazolines of formula (8) by introduction of a group R¹. This can be done by treating (8) with a base and then subsequently with an electrophile capable of introducing the group FV. A typical electrophile could be a halide such as R¹—C¹, R¹—Br, or A typical base could be n.butyl-lithium or mesityl-lithium. The Z group can be a protecting group such as Me₃Si or tBuOCO, which can be removed if desired, and a different Z group can be attached as described above if so desired.

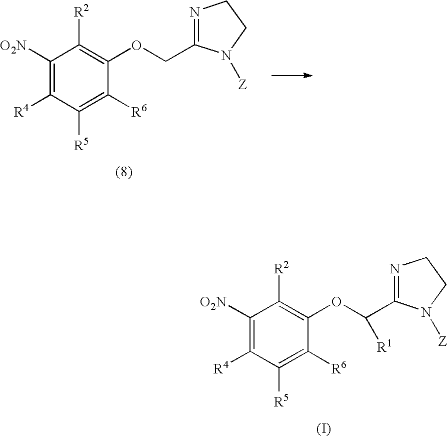

Compounds of formula (2), (3), (5), (7), (8), (9) and (10) are known compounds or may be obtained readily from known compounds using processes that are routine in the art and with which the skilled man will be familiar.

Nitriles of formula (9), and especially nitriles of formula (9a) wherein L₁ is an alkylsulfonyl or an arylsulfonyl group, may be obtained from the corresponding aldehydes by first treating with an alkali cyanide or with trimethylsilylcyanide to form a cyanohydrin of formula (12) and then treating the cyanohydrin (12) with an alkylsulfonylchloride or an arylsulfonylchloride and a base like triethylamine or pyridine to form the sulfonylcyanide (9a). The preparation of such nitriles of the formula (9) in their chiral form is described for examples in Chemische Berichte, 126, 779 (1993).

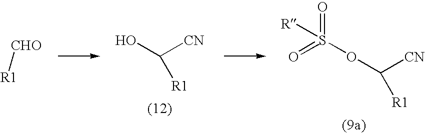

The compounds of the formula (4), wherein R¹, R², R⁴, R⁵ and R⁶ are as described in formula (I) above, have been specifically designed as intermediates for the synthesis of the compounds of the formula (I) and form yet another aspect of this invention. Table 4 below shows characterising data for illustrative examples of such intermediates.

TABLE 4
Characterising data for intermediates

| | |
|---|---|
| | (4) |
| 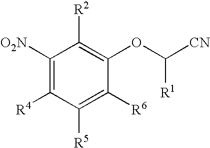 | |

| Compound | | | | | | Physical |
|---|---|---|---|---|---|---|
| No | R¹ | R² | R⁴ | R⁵ | R⁶ | data*) |
| 4.001 | C₂H₅ | CH₃ | H | H | H | 60-62 |
| 4.002 | i-C₃H₇ | CH₃ | H | H | H | oil |
| 4.003 | c-C₃H₇ | CH₃ | H | H | H | oil |
| 4.004 | n-C₃H₇ | CH₃ | H | H | H | 65-67 |
| 4.005 | n-C₄H₉ | CH₃ | H | H | H | oil |
| 4.006 | C₂H₅ | Cl | H | H | H | 69-71 |
| 4.007 | C₃H₇ | Cl | H | H | H | 76-78 |
| 4.008 | CH₂OCH₃ | CH₃ | H | H | H | oil |
| 4.009 | i-C₃H₇ | Cl | H | H | H | oil |
| 4.010 | c-C₃H₇ | Cl | H | H | H | oil |
| 4.011 | n-C₃H₇ | NO₂ | H | H | H | oil |
| 4.012 | CH₂OCH₃ | Cl | H | H | H | white solid |

*)¹H-NMR (CDCl₃) of selected compounds:
Compound No
4.002 ¹H-NMR (CDCl₃) 1.25, d, 3H; 1.29, d, 3H; 2.35-2.45, m, 1H; 2.4, s, 3H, 4.63, d, 1H, 7.22, d, 1H, 7.35, t, 1H, 7.55, d, 1H.
4.003 ¹H-NMR (CDCl₃) 0.65-0.75, m, 2H; 0.82-0.92, m, 2H; 1.55-1.67, m, 1H; 2.42, s, 3H, 4.55, d, 1H, 7.20, d, 1H, 7.35, t, 1H, 7.57, d, 1H.
4.005 ¹H-NMR (CDCl₃) 0.97, t, 3H; 1.40-1.55, m, 2H; 1.59-1.70, m, 2H; 2.10-2.20, m, 2H, 2.40, s, 3H, 4.80, dd, 1H, 7.21, d, 1H, 7.35, t, 1H, 7.55, d, 1H.
4.008 ¹H-NMR (CDCl₃) 2.41, t, 3H; 3.55, s, 3H; 3.90-4.0, m, 2H; 4.90-4.95, dd, 1H; 7.25, d, 1H; 7.35, t, 1H, 7.60, d, 1H.
4.009 ¹H-NMR (CDCl₃) 1.25, d, 3H; 1.28, d, 3H; 2.48-2.50, m, 1H; 4.65, d, 1H; 7.34, d, 1H; 7.43, t, 1H, 7.56, d, 1H.
4.010 ¹H-NMR (CDCl₃) 0.65-0.80, m, 2H; 0.80-0.94, m, 2H; 1.59-1.69, m, 1H; 4.60, d, 1H; 7.35, d, 1H; 7.42, t, 1H, 7.59, d, 1H.

The compounds of formula (I) can be used to combat and control infestations of insect pests such as Lepidoptera, Diptera, Hemiptera, Thysanoptera, Orthoptera, Blattodea, Coleoptera, Siphonaptera, Hymenoptera and Isoptera and also other invertebrate pests, for example, acarids. Insects and acarids are hereinafter collectively referred to as pests.

By the terms “combat” or “combating” it is meant that compounds of formula (I) may be used to prevent or inhibit infestation by a pest of a crop or locus of a crop. Levels of infestation may be measured by any appropriate method known in the art. An inhibition of infestation is observed where the level of infestation is lower in a crop/locus of a crop treated with a compound of formula (I) in comparison to the level of infestation observed or predicted in a crop/locus of a crop that has not been treated with a compound of formula (I).

By the terms “control” or “controlling” it is meant that, pests are repelled, are unable to feed, are unable to reproduce, and/or are killed. Thus the method of the invention may involve the use of an amount of the active ingredient that is sufficient to repel said pests (i.e a repellently effective amount of active ingredient), an amount of the active ingredient that is sufficient to stop pests feeding, an amount of the active ingredient that is sufficient to inhibit reproduction (e.g. by inhibiting oviposition or ovulation, or by mediating an ovicidal effect), or it may involve the use of an insecticidally-, nematocidally- or molluscidally-effective amount of active ingredient (i.e. an amount sufficient to kill said pests), or the method of the invention may involve any combination of the above effects.

The pests which may be combated and controlled by the use of the invention compounds include those pests associated with agriculture (which term includes the growing of crops for food, fuel, and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

For the avoidance of doubt, whilst pests may be controlled though repellent and/or ovicidal etc. modes of action and/or through killing the pest, it is particularly preferred that the compounds of formula (I) act through killing the pest.

Examples of pest species which may be controlled by the compounds of formula (1) include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locustamigratoria (locust), Chortiocetes terminifera (locust), Diabrotica spp. (rootworms), Panonychus u/ml (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice).

The invention therefore provides a method of combating and controlling an insect, or acarid pest, which comprises applying a compound of formula (I) or a composition containing a compound of formula (I) to said pest, a locus of said pest, or to a plant susceptible to attack by said pest.

In preferred embodiments compounds of formula (I) and compositions containing such compounds are used in methods of controlling and combating an insects in the orders Hemiptera, Lepidoptera, Coleoptera, Thysanoptera, Diptera, Blattodea, Isoptera, Siphonaptera, Hymenoptera, and/or Orthoptera, and in particular insects in the orders Hemiptera, Lepidoptera, Coleoptera, Thysanoptera, Blattodea, Isoptera, Siphonaptera, Hymenoptera, and/or Orthoptera. It is particularly preferred that compounds of formula (I), and composition containing these compounds are used against Hemipteran insects.

The term “plant” as used herein includes seeds, seedlings, bushes and trees. In order to apply a compound of formula (I) as an insecticide or acaricide to a pest, a locus of pest, or to a plant susceptible to attack by a pest, the compound is usually formulated into a composition which includes, in addition to the compound of formula (I), a suitable inert diluent or carrier and, optionally, a surface active agent (SFA). Suitable inert diluents or carriers are described herein, for example with respect to certain formulation types, and thus the term includes solid diluents, inorganic water soluble salts, water-soluble organic solids and the like as well as simple diluents such as, for example, water and/or oils. SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal or acaricidal, composition comprising an insecticidally or acaricidally effective amount of a compound of formula (I) and a suitable carrier or diluent therefor. The composition is preferably an insecticidal or acaricidal composition.

In a still further aspect the invention provides a method of combating and controlling pests at a locus which comprises treating the pests or the locus of the pests with an effective amount of a composition as described hereinbefore. Such compositions are preferably used against insects or acarids.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the pesticidally active ingredient i.e. a compound of formula (I).

Dustable powders (DP) may be prepared by mixing the pesticidally active ingredient with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing the pesticidally active ingredient with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing the pesticidally active ingredient with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of the pesticidally active ingredient and one or more powdered solid diluents or carriers, or from pre-formed blank granules by absorbing the pesticidally active ingredient (or a solution thereof, in a suitable agent) in a porous granular material (such as pumice, attapulgite clays, fuller's earth, kieselguhr, diatomaceous earths or ground corn cobs) or by adsorbing the pesticidally active ingredient (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving the pesticidally active ingredient in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving the pesticidally active ingredient in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining the pesticidally active ingredient either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. The pesticidally active ingredient is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of the pesticidally active ingredient. SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, the pesticidally active ingredient may be dry milled and added to water, containing agents hereinbefore described, to produce the desired end product.

Aerosol formulations comprise the pesticidally active ingredient and a suitable propellant (for example n-butane). The pesticidally active ingredient may also be dissolved or dispersed in a suitable medium (for example water or a water miscible liquid, such as n-propanol) to provide compositions for use in non-pressurised, hand-actuated spray pumps.

The pesticidally active ingredient may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains the pesticidally active ingredient and, optionally, a carrier or diluent therefor. The polymeric shell may be produced by either an interfacial polycondensation reaction or by a coacervation procedure. The compositions may provide for controlled release of the compound of formula (I) and they may be used for seed treatment. The pesticidally active ingredient may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of the pesticidally active ingredient). Such additives include surface active agents, spray additives based on oils, for example certain mineral oils or natural plant oils (such as soy bean and rape seed oil), and blends of these with other bio-enhancing adjuvants (ingredients which may aid or modify the action of the pesticidally active ingredient).

In particularly preferred embodiments, compounds of formula I will be formulated as an EC or EW formulation.

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

Compounds of formula (I) may be applied by any of the known means of applying pesticidal compounds. For example, they may be applied, formulated or unformulated, to the pests or to a lbws of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or they may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

Compounds of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

Compounds of formula (I) (either individually or in combination with each other) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); and/or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, spinosad or azadirachtin;
h) Hormones or pheromones;
i) Organochlorine compounds such as endosulfan, benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds such as imidacloprid, thiacloprid, acetamiprid, nitenpyram, dinotefuran, thiamethoxam or sulfoxaflor;
m) Diacylhydrazines, such as tebufenozide, chromafenozide or methoxyfenozide;
n) Diphenyl ethers, such as diofenolan or pyriproxifen;
o) lndoxacarb;

### p) Chlorfenapyr;

### q) Pymetrozine;

r) Tetronic acids such as spirotetramat, spirodiclofen or spiromesifen;
s) Spinosyns, such as spinosad, or spinetoram; or t) Anthranilic diamides such as flubendiamide, Cyazypyr™ or Rynaxypyr™.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoro-methyl-benzimidazole-1-sulphonamide, α-[N-(3-chloro-2,6-xylyl)-2-methoxy-acetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)-propionamide (AC382042), N-(2-methoxy-5-pyridyl)-cyclo-propane carboxamide, acibenzolar (CGA245704), alanycarb, aldimorph, anilazine, azaconazole, azoxystrobin, benalaxyl, benomyl, bi-loxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, diclomezine, dicloran, diethofencarb, difenoconazole, difenzoquat, diflumetorim, O,O-di-iso-propyl-5-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-N-([methyl(methyl-thioethylideneamino-oxycarbonypaminoithio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-iso-propyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-Al, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrroInitrin, quaternary ammonium compounds, quinomethionate, quinoxyfen, quintozene, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

Various aspects and embodiments of the present invention will now be illustrated in more detail by way of example. It will be appreciated that modification of detail may be made with out departing from the scope of the invention.

For the avoidance of doubt, where a literary reference, patent application, or patent, is cited within the text of this application, the entire text of said citation is herein incorporated by reference.

### EXAMPLES

### Example 1 2-O-(Chloro-3-nitro-phenoxy)-propyl]-4,5-dihydro-1H-imidazole

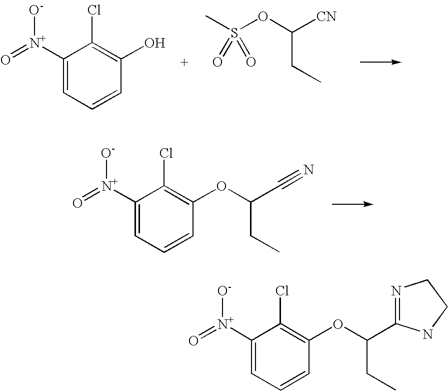

2-chloro-3-nitro-phenol (1.0 g, 5.8 mmol) was dissolved in 3 ml N-methyl-pyrolidone at 30° C. The resultant yellow solution was degassed with nitrogen. After addition of cesium carbonate (1.69 g, 5.2 mmol) the suspension was stirred for 1 hour. 2-(methanesulfonyloxy)-butyronitrile (1.41 g, 8.6 mmol) dissolved in 2 ml N-methyl-pyrolidone and potassium iodide (0.096 g, 0.1 mmol) were then added and the reaction mixture was heated at 60° C. After 2.5 hours an additional portion of N-methyl-pyrolidone (2 ml) was added and the reaction mixture was stirred for further 15 hours. The reaction was monitored by TLC. On completion, the mixture was poured into 50 ml water (0° C.) and extracted with t-butyl-methylether. The organic layer was separated and washed with water and brine and dried with Na₂SO₄. The crude material was purified on silica gel by flash chromatography (ethylacetate/cyclohexane, 1:6) to give 2-(2-chloro-3-nitro-phenoxy)-butyro nitrile as yellow solid (M.p. 69-71° C.). ¹H-NMR (CDCl₃) 1.26, t, 3H, 2.21 m, 2H, 4.81 dd, 1H, 7.35 d, 1H, 7.42 t, 1H, 7.56 d, 1H.

A mixture of 2-(2-chloro-3-nitrol-phenoxy)-butyronitrile (700 mg, 2.9 mmol), ethylene diamine (0.78 ml, 11.6 mmol) and sodium tetrasulfide (30 mg, 0.2 mmol) was stirred for 9 hrs at 60° C., then cooled to room temperature. The solvent was removed by distillation and 40 ml cold water (0° C.) was added to the reaction mixture. The resultant suspension was extracted with ethyl acetate. The organic layer was separated and dried with Na₂SO₄. The crude material was purified on silica gel by filtration (CH₂Cl₂/MeOH 9:1) to give 2-[1-(2-Chloro-3-nitro-phenoxy)-propyl]-4,5-dihydro-1H-imidazole as yellow solid (M.p. 100-103° C.). ¹H-NMR (CDCl₃) 1.10, t, 3H, 1.95-2.15, m, 2H, 3.47-3.80, m, 4H, 4.90 dd, 1H, 7.29-7.35 m, 2H, 7.39 dd, 1H.

### Example 2 2-[1-(2-Chloro-3-nitro-phenoxy)-but-3-enyl]-4,5-dihydro-1H-imidazole

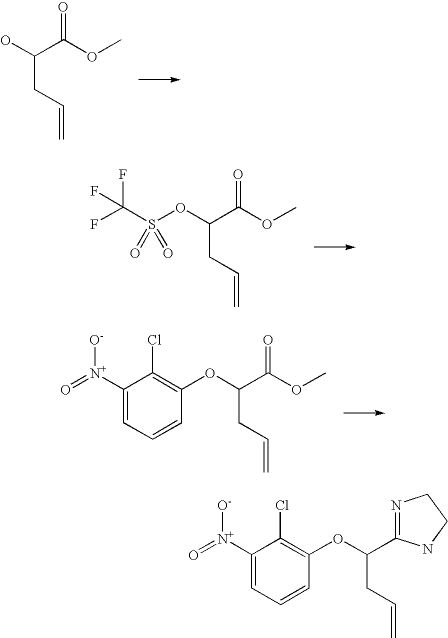

A solution of 2-hydroxy-pent-4-enoic acid methyl ester (1.00 g, 7.68 mmol) and 2,6-lutidine (1.03 ml, 8.91 mmol) in dichloromethane (15 ml) was stirred at 0° C. under nitrogen. Trifluoromethanesulfonic anhydride (1.42 ml, 8.45 mmol) was added dropwise, keeping the temperature of the reaction mixture below 10° C. After the addition the reaction mixture was stirred for a further 20 minutes at 0° C. TIc (5% acetone in toluene) showed the reaction to be complete. The mixture was shaken between tBuOMe and HCl (1M aq.), washed with NaHCO₃ (satd ag), dried with magnesium sulfate, and evaporated to yield crude material, which was chromatographed on silica with 10% EtOAc/heptane to yield pure 2-trifluoromethanesulfonyloxy-pent-4-enoic acid methyl ester.

2-chloro-3-nitro-phenol (2.0 g, 11.5 mmol) was dissolved in 10 ml N-methyl-pyrolidone at 20° C. under nitrogen. After addition of cesium carbonate (4.13 g, 12.7 mmol) the suspension was stirred for 30 minutes. 2-trifluoromethanesulfonyloxy-pent-4-enoic acid methyl ester (3.32 g, 12.7 mmol) and potassium iodide (0.1 g, 0.6 mmol) were then added and the reaction mixture was stirred for 30 minutes at 20° C. The reaction was monitored by TLC. On completion, the mixture was poured into water (0° C.) and extracted with ethylacetate. The organic layer was separated and washed with water and brine and dried with MgSO₄. The crude material was purified on silicagel by flash chromatography (eluent: methylene dichloride) to give 2-(2-chloro-3-nitro-phenoxy)-pent-4-enoic acid methyl ester as colourless oil.

¹H-NMR (CDCl₃) 2.78-2.85, m, 2H, 3.78, s, 3H, 4.75, dd, 1H, 5.16-5.29, m, 2H, 5.88-5.99, m, 1H, 6.99 d, 1H, 7.30, t, 1H, 7.41, d, 1H.

Ethylenediamine (0.53 g, 8.8 mmol) solved in 2 ml toluene is added dropwise to a stirred solution of trimethylaluminum (0.63 g, 8.8 mmol) in 2 ml toluene, so that the temperature does not exceed 10° C. After stirring for one hour a solution of 2-(2-chloro-3-nitro-phenoxy)-pent-4-enoic acid methyl ester (1.25 g, 4.4 mmol) in 2 ml toluene is gradually added. The reaction mixture is refluxed for 3 hours. After cooling, the solution is treated dropwise with methanol and methylene chloride. Then the reaction mixture is poured into cold water and extracted with methylene chloride. The organic layer was dried over MgSO₄, filtered and concentrated. The crude material was then treated with aq HCl and extracted with ethylacetate to remove impurities. The water layer was basified with Na₂CO₃ (satd aq) and the resultant suspension extracted with ethyl acetate. After drying over MgSO₄, filtering and concentration yellowish oil was obtained. This was treated with heptane to yield pure 2-[1-(2-chloro-3-nitro-phenoxy)-but-3-enyl]-4,5-dihydro-1H-imidazole (Mp: 137-142).

¹H-NMR (CDCl₃) 2.62-2.85, m, 2H, 3.30-4.00, m (broad), 4H, 4.92-5.05, dd, 1H, 5.10-5.30, m, 2H, 5.81-6.0, m, 1H, 7.2-7.5, m, 3H.

Example 3 2-[2-Methoxy-1-(2-methyl-3-nitro-phenoxy)-ethyl]-4,5-dihydro-1H-imidazole

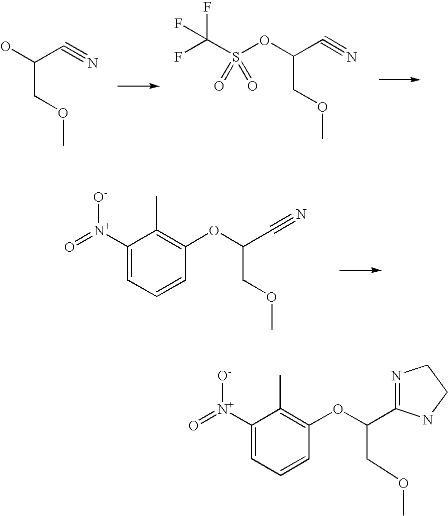

Trifluoromethanesulfonic anhydride (19.9 ml, 118 mmol) was added dropwise with stirring to a solution of 2-hydroxy-3-methoxy-propionitrile (7.87 g, 7.8 mmol) and 2,6-lutidine (13.7 ml, 118 mmol) in dichloromethane (ca 80 ml) with a cold bath keeping the temperature of the reaction mixture between −5° C. and −20° C. After the addition was complete the mixture was stirred for 15 minutes at 0° C. Water (10 ml) was added and the temperature rose to 7° C. After 15 minutes the mixture was shaken between tBuOMe and water, washed with HCl (1M) and NaHCO₃ (satd), dried with magnesium sulfate, and concentrated under vacuum to gove crude material, which was chromatographed on silica with EtOAc and hexane to yield pure trifluoro-methanesulfonic acid 1-cyano-2-methoxy-ethyl ester.

2-Methyl-3-nitro-phenol (1.0 g, 6.53 mmol) was dissolved in 10 ml N-methyl-pyrolidone at 20° C. The resultant solution was degassed with nitrogen. After addition of cesium carbonate (3.19 g, 9.8 mmol) the suspension was stirred for 30 minutes. Trifluoro-methanesulfonic acid 1-cyano-2-methoxy-ethyl ester (2.28 g, 9.8 mmol) and potassium iodide (0.054 g, 0.05 mmol) were then added and the reaction mixture was heated at 100° C. for 3 minutes under micro. wave irradiation (Initiator™ Sixty, Biotage). After cooling for two minutes the reaction mixture was once again heated under microwave irradiation at 100° C. for 3 minutes. On completion, the mixture was poured into cold aq NaOH (0° C.) and extracted three times with diethyl ether. The organic layers were combined and washed with water and brine and dried with Na₂SO₄. The crude material was purified on silicagel by flash chromatography (ethylacetate/cyclohexane, 1:9) to give 3-methoxy-2-(2-methyl-3-nitro-phenoxy)-propionitrile as oil.

¹H-NMR (CDCl₃) 2.41, t, 3H, 3.55, s, 3H, 3.90-4.0, m, 2H, 4.90-4.95, dd, 1H, 7.25, d, 1H, 7.35, t, 1H, 7.60, d, 1H.

In a microwave vial a mixture of 3-methoxy-2-(2-methyl-3-nitro-phenoxy)-propionitrile (700 mg, 2.9 mmol), ethylene diamine (2 ml) and sodium tetrasulfide (10 mg, 0.07 mmol) was heated for 3 minutes at 70° C. under micro wave irradiation, then cooled for about 1 minute, and again heated to 70° C. for three minutes (Initiator™ Sixty, Biotage). After a further short cooling period (ca 1 minute) the reaction mixture was once again heated to 70° C. for three minutes. On completion, the reaction mixture was poured into cold water (0° C.). The resultant suspension was extracted with dichloromethane. The organic layer was separated and dried with Na₂SO₄. The crude material was purified on silicagel by flash chromatography (CH₂Cl₂/MeOH 9:1) to give 2-[2-methoxy]-1-(2-methyl-3-nitro-phenoxy)-ethyl]-4,5-dihydro-1H-imidazole as yellow solid (M.p. 65-72° C.).

¹H-NMR (CDCl₃): 2.41, s, 3H, 3.44, s, 3H, 3.56-3.75, m, 4H, 3.81-3.92, m, 2H, 5.02-5.06, dd, 1H, 7.15-7.31, m, 2H, 7.45, d, 1H.

### Example 4

### Biological Efficacy

This Example illustrates the pesticidal/insecticidal properties of compounds of formula (I). Compounds are identified with respect to the numbers allocated in the tables of characterising data (i.e. Tables 3 and 4). Tests against various pest species were performed as described below.

### 4.1 Heliothis Virescens (Tobacco Budworm):

Eggs (0-24 h old) were placed in 24-well microtiter plate on artificial diet and treated with test solutions at an application rate of 200 ppm by pipetting. After an incubation period of 4 days, samples were checked for egg mortality, larval mortality, and growth regulation. The following compounds gave 100% control: 1.003 and 1.009. The following compounds gave 80% control: 1.001, 1.004, 1.005, 1.008 and 1.016. The following compounds gave 50% control: 1.013 and 1.017.

### 4.2 Myzus Persicae (Green Peach Aphid):

Sunflower leaf discs were placed on agar in a 24-well microtiter plate and sprayed with test solutions at an application rate of 200 ppm. After drying, the leaf discs were infested with an aphid population of mixed ages. After an incubation period of 6 DAT, samples were checked for mortality. The following compounds gave 100% control: 1.001, 1.002, 1.003, 1.004, 1.005, 1.006, 1.008, 1.009, 1.012, 1.013, 1.014, 1.016, 1.017 and 1.018. The following compounds gave 80% control: 1.007 and 1.011. The following compound gave 50% control: 1.010

### 4.3 Myzus Persicae (Green Peach Aphid):

Roots of pea seedlings, infested with an aphid population of mixed ages, were placed directly in the test solutions of 24 ppm. 6 days after introduction, samples were checked for mortality. The following compounds gave 100% control: 1.004 and 1.012. The following compounds gave 80% control: 1.001, 1.002, 1.009, 1.013 and 1.016. The following compound gave 50% control: 1.003.

### 4.4 Tetranychus Urticae (Two-Spotted Spider Mite):

Bean leaf discs on agar in 24-well microtiter plates wer sprayed with test solutions at an application rate of 200 ppm. After drying, the leaf discs are infested with mite populations of mixed ages. 8 days later, discs are checked for egg mortality, larval mortality, and adult mortality. The following compounds gave 100% control: 1.001, 1.002, 1.003, 1.004, 1.006, 1.008, 1.009, 1.010, 1.012, 1.013, 1.014, 1.016, 1.017 and 1.018. The following compound gave 50% control: 1.011.

1. A method of combating and/or controlling an insect or acarid pest, which comprises applying to said pest, or to the locus of said pest, or to a plant susceptible to attack by said pest, a compound of formula (I): 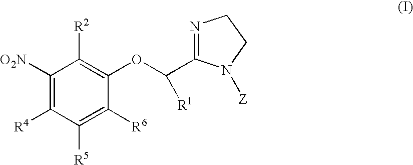
or salt or N-oxide thereof, wherein:
R¹ is (i) an optionally substituted C₁₋₆ alkyl, (ii) an optionally substituted C₂₋₆ alkenyl, (iii) an optionally substituted C₃₋₆ cycloalkyl, (iv) an optionally substituted C₃₋₆ cycloalkenyl, or (v) an optionally substituted C₂₋₆ alkynyl;
R² is C₁₋₅ alkyl, C₁₋₅ haloalkyl, C₂₋₅ alkenyl, C₂₋₅ haloalkenyl, C₂₋₅ alkynyl, C₃₋₆ cycloalkyl, C₁₋₅ alkoxy, C₁₋₅ haloalkoxy, C₁₋₃ alkoxy(C₁₋₃)alkyl, C₁₋₅ alkylthio, C₁₋₅ haloalkylthio, C₁₋₅ alkyl sulfonyl, C₁₋₅ alkylsulfinyl, C₁₋₅ haloalkylsulfonyl, C₁₋₅ haloalkylsulfinyl, cyano, nitro, halogen, or formyl;
R⁴ is hydrogen, methyl or halogen;
R⁵ is hydrogen, methyl or halogen;
R⁶ is hydrogen, methyl, or halogen;
Z is hydrogen, hydroxy, nitro, cyano, rhodano, formyl, G-, G-S—, G-S—S—, G-A-, G-O—, G-A-O—, G-X-A-O—, R⁷R⁸N—, R⁷R⁸N—S—, R⁷R⁸N-A-, G-O-A-, G-S-A-, (R¹⁶O)(R¹¹O)P(X)—, (R¹⁰O)R¹¹S)P(X)—, (R¹⁰O)(R¹¹)P(X)—, b)(R¹⁰S)(R¹¹S)P(X)—, (R¹⁶O)(R¹⁴R¹⁵N)P(X)—, (R¹¹)(R¹⁴R¹⁵N)P(X)—, (R¹⁴R¹⁵N)(R¹⁶R¹⁷N)P(X)—, G-N═CH—, G-O—N═CH—, NEC-N═CH—, or Z is group of formula (II) 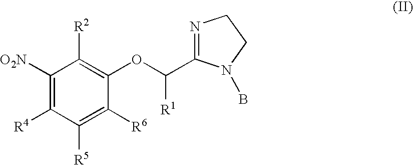
wherein B is S—, S—S—, S(O)—, C(O)—, or (CHA-, n is an integer from 1 to 6; R¹, R², R⁴, R⁵ and R⁶ are as defined above, and
G is optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted C₃₋₇ cycloalkenyl, optionally substituted aryl, optionally substituted heteroaryl or optionally substituted heterocyclyl;
A is S(O), SO₂, C(O) or C(S);
R⁷ and R⁸ are each independently hydrogen or G; or R⁷ and R⁸ together with the N atom to which they are attached form a group N═CR¹²R¹³; or R⁷ and R⁸ together with the N atom to which they are attached form a five, six or seven-membered heterocyclic ring, which heterocyclic ring optionally contains one or two further heteroatoms selected from O, N or S, and is optionally substituted by one or two C₁₋₆ alkyl groups;
R¹⁰ and R¹¹ are each independently C₁₋₆ alkyl, benzyl or phenyl where the phenyl group is optionally substituted with halogen, nitro, cyano, C₁₋₃ alkyl, C₁₋₃ haloalkyl, C₁₋₃ alkoxy, C₁₋₃ haloalkoxy;
R¹², R¹³, R¹⁴, R¹⁵, R¹⁶ is and R¹⁷ are each independently hydrogen or C₁₋₆ alkyl;
X is O or S.

2. The method of claim 1, wherein said substitution of R¹ in any one of (i) to (v) is selected from one or more of: halogen; nitro; cyano; rhodano; carboxy; formyl; formyloxy; formylamino; optionally substituted C₃₋₇ cycloalkyl, said substitution being selected from one or more of halogen, hydroxy, nitro, cyano, rhodano, C₁₋₃ alkyl, C₁₋₃ haloalkyl and C₁₋₃ alkoxy; optionally substituted C₃₋₇ cycloalkenyl, said substitution being selected from one or more of halogen, hydroxy, nitro, cyano, rhodano, C₁₋₃ alkyl, C₁₋₃ haloalkyl, and C₁₋₃ alkoxy; optionally substituted aryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, and C₁₋₄ alkoxycarbonyl; optionally substituted heteroaryl, said substitution being selected from one or more of halogen, hydroxy, nitro, cyano, rhodano, C₁₋₃ alkyl, C₁₋₃ haloalkyl, C₁₋₃ alkoxy, C₁₋₃ haloalkoxy, C₁₋₃ alkylthio, C₁₋₃ alkylsulfinyl, C₁₋₃ alkylsulfonyl, C₁₋₃ haloalkylthio, C₁₋₃ alkoxycarbonyl, carbamoyl, C₁₋₄ alkylaminocarbonyl and di-C₁₋₄ alkylaminocarbonyl; optionally substituted heterocyclyl, said substitution being selected from one or more of halogen, hydroxy, nitro, cyano, rhodano, C₁₋₃ alkyl, C₁₋₃ haloalkyl, and C₁₋₃ alkoxy; G-O—; G-S—; G-A-; G-A-O—; G-A-S—; R⁷R⁸N—; R⁷R⁸N-A-; G-O-A-; G-S-A-; G-A-NR⁹—; R⁷R⁸N-A-NR⁹—; and G-O-A-NR⁹—, and wherein G, A, R⁷, R⁸, and R⁹ are as defined in claim 1.

3. The method according to claim 1 wherein R² is methyl, fluoromethyl, difluoromethyl, trifluoromethyl, fluoro, chloro or bromo.

4. The method according to claim 1 wherein R⁴, R⁵ and R⁶ are each hydrogen.

5. A compound of formula (IA) 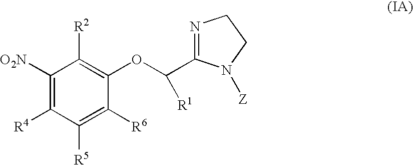
or salt or N-oxide thereof, wherein:
R¹ is (i) substituted C₁₋₂ alkyl provided said substitution is not a hydroxyl group (ii) optionally substituted C₃₋₆ alkyl, (iii) optionally substituted C₂₋₆ alkenyl, (iv) optionally substituted C₃₋₆ cycloalkyl, (v) optionally substituted C₃₋₆ cycloalkenyl, or (vi) optionally substituted C₂₋₆ alkynyl; and R², R⁴, R⁵, R⁶ and Z are as defined in claim 1.

6. An insecticidal or acaricidal composition comprising the compound of formula (I) as defined in claim 5.

7. A process for the preparation of a compound of formula (I) as defined in claim 1, which comprises reacting a compound of the formula (4) 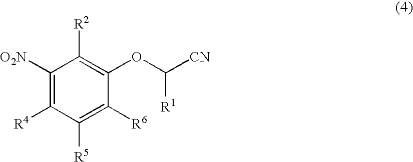
with a diamine of the formula (5)
H₂N—C₂H₄—NHZ (5),
in the presence of a catalyst, wherein R¹, R², R⁴, R⁵, R⁶ and Z are as defined in claim 1.

8. A compound of the formula (4) 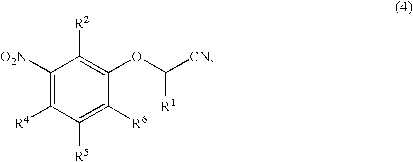
wherein R¹, R², R⁴, R⁵ and R⁶ are as defined in claim 1.
